(19) United States
(12) Patent Application Publication
(10) Pub. No.: US 2019/0350699 A1
(43) Pub. Date: Nov. 21, 2019

(54) THERMOELECTRICALLY-POWERED DEVICE FOR THERAPEUTIC PRESBYOPIA VISION CORRECTION

(71) Applicant: Quantum Medical Innovations, LLC, Jacksonville, FL (US)

(72) Inventors: Glen Jorgensen, Jacksonville, FL (US); Steven Reeser, Jacksonville, FL (US)

(21) Appl. No.: 16/380,756

(22) Filed: Apr. 10, 2019

Related U.S. Application Data
(60) Provisional application No. 62/672,140, filed on May 16, 2018.

(51) Int. Cl. A61F 2/16; G02C 7/08

(52) U.S. Cl. CPC A61F 2/1613; G02C 7/04; G02C 7/083

(57) ABSTRACT

A therapeutic device for correcting presbyopia in an eyeball having a ciliary muscle and a plurality of suspensory ligaments connecting the ciliary muscle to a lens includes an over-mold that mirrors a contact lens and fits against the eyeball. The over-mold encapsulates a micro thermoelectric generator (“μTEG”) that powers an electrical disc sub-assembly configured for sensing an activation of the ciliary muscle from a relaxed state, in which the ligaments are pulled taut and the lens is stretched into a flat position for distant focus, toward a contracted state, in which the ligaments become less taut and the lens moves into a rounded position for nearby focus. When the ciliary muscle is contracted, the sub-assembly stimulates the ciliary muscle with a minute electric muscle stimulation (“EMS”) to further contract the ciliary muscle, thereby further relaxing the ligaments to further improve nearby focus. Other embodiments are disclosed.

### REFERENCE TO PENDING PRIOR PATENT APPLICATION

This application claims the benefit under 35 U.S.C. 119(e) of U.S. Provisional Patent Application No. 62/672,140, filed May 16, 2018 by Glen Jorgensen and Steven Reeser, for “THERMOELECTRICALLY-POWERED DEVICE FOR THERAPEUTIC PRESBYOPIA VISION CORRECTION,” which patent application is hereby incorporated herein by reference.

### BACKGROUND

FIG. 1 illustrates a cross-sectional view of a human eyeball 50 and its various components, while FIGS. 2-3 provide partial views of a ciliary-zonula complex 58 of the human eye 50 for use in controlling optical accommodation. Optical accommodation is the process by which the eye adjusts its focal distance to maintain a clear image of objects at varying distances. Optical accommodation is controlled by contractions of a ciliary muscle 52, which encircles the eye's elastic lens 54 and applies a force around the lens 54 during ciliary muscle contractions that change the radial shape of the elastic lens 54, thereby adjusting the focal point of the lens 54.

As shown in the electron microscopic image of FIG. 2, the lens 54 is suspended from the ciliary muscle 52 by suspensory ligaments 56. (Gregory, Richard Langton (1977), Eye and Brain: the psychology of seeing (3rd ed. rev. and update. ed.), New York; Toronto: McGraw-Hill. ISBN 0070246653.) These suspensory ligaments 56 are also referred to herein as the “zonula” or “zonules” 56, which are held under tension by the ciliary muscle 52, as illustrated in FIG. 3. The combination of the zonula 56 and the ciliary muscle 52 forms the “ciliary-zonula complex” 58.

FIGS. 4A-4B illustrate the process of optical accommodation via the ciliary-zonula complex 58. Specifically, FIG. 4A illustrates a geometry of the eye 50, as trained upon a distant object. In this geometry, the suspensory ligaments or zonules 56 (FIGS. 1-3) are tensioned and pulled tauter by the relaxed ciliary muscle 52 in a relaxed state 64, causing the lens 54 to stretch into a flat position for distant focus. The tension is released by contraction of the ciliary muscle 52 to a contracted state 66, as shown in FIG. 4B, to allow the lens 54 to become more rounded, changing the refractive index for close vision or nearby focus. The ciliary muscle 52, which is positioned outside the zonula 56, must be circumferential, contracting like other body sphincters to make the inside diameter smaller and to slacken the tension of the zonula 56, which normally pull outwards upon the lens 54. This is consistent with the fact that our eyes seem to be in the “relaxed” state when focusing at infinity, and also explains why no amount of effort seems to enable a myopic (near-sighted) person to see farther away.

Presbyopia is a condition associated with aging of the eye and results in a progressively worsening ability to focus clearly on close objects. (“Facts About Presbyopia”, NEI, October 2010, Archived from the original on 4 Oct. 2016, Retrieved 11 Sep. 2016.) Symptoms include difficulty reading small print, having to hold reading material farther away, headaches, and eyestrain. Presbyopia is a natural part of the aging process. It is due to (1) a hardening of the lens 54 of the eye 50, causing the eye 50 to focus light behind, rather than on, the retina 59 (FIG. 1) when looking at close objects, and (2) a relaxation of the elasticity of the ciliary muscle 52 and the zonules 56, or the ciliary-zonula complex 58, during optical accommodation of the refractive index of the lens 54.

Diagnosis of presbyopia is by an eye examination. Treatment typically involves eyeglasses having a higher focusing power in the lower portion of the lens. Off-the-shelf reading glasses may be sufficient for some.

Many people with myopia, or near-sightedness, can read comfortably without eyeglasses or contact lenses, even after age forty. However, their myopia does not disappear and the long-distance visual challenges remain. Myopes considering refractive surgery are generally advised that surgically correcting their near-sightedness may be at a disadvantage after age forty, when the eyes become presbyopic and lose their ability to accommodate or change focus, because they will then need to use glasses for reading. Myopes with astigmatism find near vision better, though not perfect, without glasses or contact lenses when presbyopia sets in, but the more astigmatism, the poorer the uncorrected near vision becomes.

Corrective lenses provide a range of vision correction, some as high as +4.0 diopter. Some with presbyopia choose varifocal or bifocal lenses to eliminate the need for a separate pair of reading glasses, though specialized preparations of varifocals or bifocals usually require the services of an optometrist. Some newer bifocal or varifocal spectacle lenses attempt to correct both near and far vision with the same lens.

Contact lenses can also be used to correct the focusing loss that comes along with presbyopia. Multifocal contact lenses can be used to correct vision for both the near and the far. Some people choose contact lenses to correct one eye for near and one eye for far with a method called monovision.

Corneal inlays surgically implanted into the cornea 61 (FIG. 3) are another current treatment option for presbyopia. Corneal inlays typically are implanted in the nondominant eye to minimize impact to binocular uncorrected distance vision. Corneal inlays seek to improve near vision in one of three ways: changing the central refractive index, increasing the depth of focus through the use of a pinhole, and reshaping the central cornea.

The state-of-the-art in ophthalmic or intraocular lenses is driven by the perceived requirement that the solution is to, in some way, add a “second lens” to the eye to accommodate the required changes in the refractive index of the natural lens or to modify the natural lens in an irreversible manner. The wearer is thereafter dependent upon the modified lens correction. Generally, most users consider corrective lenses a nuisance: searching for misplaced glasses, accommodating the vision angle through bifocals to find clarity somewhere, fogging of the lenses, an uncomfortable feeling from the weight of the lenses on the nose or the ears, falling off during athletic activities, aesthetic considerations, and so on. Contact lenses and implants address many of these short-comings, but they, too, have their own set of costs and nuisance conditions.

In view of the shortcomings of any form of surgical correction or applying a second lens for vision correction, there exists a need to therapeutically strengthen the ciliary-zonula complex 58 of the natural lens in a manner that can repair and restore the focusing limitations of aging eyesight.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

One embodiment provides a therapeutic device for presbyopia vision correction of a human eyeball having a ciliary muscle, a lens, and a plurality of suspensory ligaments connecting the ciliary muscle to a periphery of the lens. The therapeutic device may include: (1) an over-mold configured to fit flush against the eyeball; (2) a micro power supply encapsulated by and secured within the over-mold; and (3) an electrical disc sub-assembly powered by the micro power supply and encapsulated by and secured within the over-mold, the electrical disc sub-assembly configured for: (a) detecting an activation of the ciliary muscle from a relaxed state in which the plurality of the suspensory ligaments are pulled taut and the lens is stretched into a flat shape for distant focus toward a contracted state in which the plurality of the suspensory ligaments relax and the lens moves into a rounded shape for nearby focus; (b) when the activation of the ciliary muscle is detected, providing an electrical muscle stimulation (EMS) to the ciliary muscle to cause the ciliary muscle to contract further, thereby further relaxing the plurality of the suspensory ligaments to improve the nearby focus; and (c) when the activation of the ciliary muscle is not detected, terminating the EMS to allow the ciliary muscle to return to the relaxed state, thereby pulling the plurality of the suspensory ligaments taut and returning the lens to the flat shape for the distant focus.

Another embodiment provides a therapeutic device for exercising a ciliary muscle of a human eyeball. The device may include an encapsulate configured to fit flush against the eyeball, the encapsulate enveloping: (1) a micro thermoelectric generator (μTEG) configured to generate power from a temperature differential between a surface temperature of the eyeball and an ambient temperature of an ambient atmosphere; and (2) an electrical disc sub-assembly coupled with and powered by the μTEG, the electrical disc sub-assembly configured to provide an electrical muscle stimulation (EMS) to the ciliary muscle.

Yet another embodiment provides a method of correcting presbyopia in a human eyeball having a ciliary muscle, a lens, and a plurality of zonules connecting the ciliary muscle to a periphery of the lens. The method may include the following steps: (1) positioning a therapeutic device for presbyopia vision correction flush against the eyeball, the therapeutic device having an encapsulate encasing a micro thermoelectric generator (μTEG) and an electrical disc sub-assembly electrically coupled with the μTEG, the electrical disc sub-assembly having a switching and amplification circuit communicatively coupled with at least one electrode; (2) operating the μTEG to generate power from a temperature differential between a surface temperature of the eyeball and an ambient temperature of an ambient atmosphere; and (3) conducting, via the switching and amplification circuit, a current flow from a power output of the μTEG to the at least one electrode to provide an electrical muscle stimulation (EMS) to the ciliary muscle.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1 illustrates a cross-sectional view of a human eyeball, detailing the components and mechanics of the eyeball;

FIG. 2 illustrates a partial view of the human eyeball, detailing a ciliary-zonula complex of the eyeball;

FIG. 3 illustrates a partial cross-sectional view of the human eyeball, detailing the ciliary-zonula complex of the eyeball;

FIGS. 4A-4B provide schematic views depicting the ciliary muscle of the human eyeball in a relaxed state for far vision and a contracted state for near vision, respectively;

FIG. 5 provides a chart summarizing the theoretical power characteristics of an exemplary Thermal Electric Generator (TEG) at varying temperature differentials;

FIG. 6 illustrates a cross-sectional view of the human eyeball, including critical dimensions for calculating a power requirement for a thermoelectrically powered therapeutic device for presbyopia vision correction;

FIGS. 7A-7D illustrate perspective, cross-sectional, partial cross-sectional, and partial views of one embodiment of a thermoelectrically powered therapeutic device for presbyopia vision correction;

FIGS. 8A-8B provide schematics illustrating the Seebeck Effect, which predicts that positive- and negative-charged materials connected to thermally conductive heat sources will generate electrical current as the heat causes the positive and negative charge carriers to move from hot to cold;

FIG. 9 illustrates a top perspective view of one embodiment of an insulated substrate printed with a thermocouple for incorporation into a micro thermoelectric generator of the therapeutic device of FIGS. 7A-7D;

FIG. 10 provides a top perspective view of one embodiment of an electrical disc sub-assembly for incorporation into the therapeutic device of FIGS. 7A-7D.

FIG. 11 provides a schematic of an IR sensor for incorporation into the electrical disc sub-assembly of FIG. 10;

FIGS. 12A-12B illustrate respective side views of the naked eyeball and of the thermoelectrically powered therapeutic device for presbyopia vision correction of FIGS. 7A-7D, as worn against the eyeball in a manner similar to a contact lens; and

FIG. 13 provides a flowchart depicting an exemplary method of using an embodiment of the therapeutic device of FIGS. 7A-7D to therapeutically stimulate the ciliary muscle 52 for presbyopia vision correction.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

### Overview

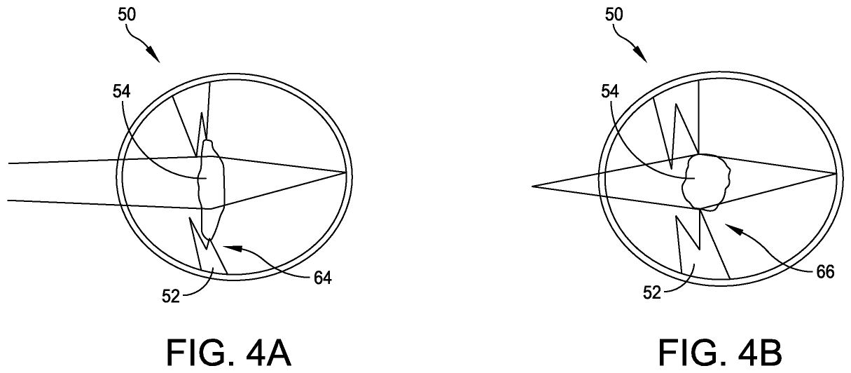

As shown in FIG. 4A, the eye's default state is to relax the ciliary muscle 52 in the relaxed state 64, which both naturally tensions the zonal fibers, or zonules 56, and flattens the lens 54 for long-distance vision. When the brain activates or triggers the requirement for near-vision, as shown in FIG. 4B, the ciliary muscle 52 contracts as a sphincter and closes in on itself into the contracted state 66, thereby releasing the tension on the zonules 56 and allowing the lens 54 to take a more rounded shape, which improves nearby focus or near-sightedness. In this contracted state 66, some sort of stimulating potential would further exercise the ciliary muscle 52, further release the tension of the zonal fibers 56, and allow the lens 54 to further increase its radius of curvature for improved near vision. As the body calls again for distant vision and the ciliary muscle 52 relaxes, the now more pliant lens 54 may resume a flatter profile than before, which tends toward improved distant vision. Exercising the lens 54 in this manner tends to improve lens elasticity and, therefore, extend its limits of vision correction for both near and far focus.

Accordingly, embodiments of the system and methods disclosed herein relate to a new, therapeutic approach to strengthening the ciliary-zonula complex 58 and exercising the natural lens 54 to improve its elasticity, combining both approaches to restore the patient's accommodation to both near- and far-sightedness in the long term, and ultimately obviating the need for corrective ophthalmic appliances of any kind. Some embodiments provide a means to strengthen the ciliary muscle 52 to tighten the complex 58 that tensions the lens for improved far-sighted accommodation. This strengthening of the ciliary muscle 52 coincidentally exercises the lens 54 to improve its elasticity, which, in turn, improves the accommodative response of the lens 54 to near-sighted requirements when the ciliary muscle 52 contracts into the contracted state 66 and releases the tension on the zonula fibers 56.

The therapeutic strengthening effect of pulsing Electrical Muscle Stimulation (EMS) has been employed on larger muscle groups in the body. This operating principle may be applied in disclosed embodiments of a device for therapeutic presbyopia vision correction to deliver a micro voltage potential to the ciliary muscle 52 through electrodes powered by a micro-sized power supply, all of which is packaged in an extended-wear, contact-lens-configuration encased in a hydrogel layer that provides a comfortable contact surface to the wearer and is commonly used in the manufacture of soft contact lenses. The therapeutic strengthening effect of pulsing EMS eventually improves the strength of the ciliary muscle 52 and the elasticity of the lens 54 such that the natural accommodation of the eye's near- and far-focus is improved and, therefore, the need for the device, and/or any other correctional ophthalmic appliance, is gradually diminished.

In some embodiments, the device may be powered by a common 9V battery. In another exemplary embodiment, the device may fit upon a contact lens-sized encapsulate. Thus, in some embodiments, the power supply may be a micro-battery, as taught in U.S. Pat. No. 9,857,608 to Jorgensen, Reeser (the “Jorgensen micro-battery”). Because the life of the Jorgensen micro-battery is typically limited to a few hours, it may be insufficient for the therapeutic manipulation of the ciliary muscle 52. Thus, some embodiments may employ a micro-sized Thermal Electric Generators (“TEG”) that uses the body's heat to perpetually power the device. One exemplary TEG is provided in U.S. Pat. No. 7,629,531 to Stark. Other applicable advancements have also been published and offer value for powering the device embodiments disclosed herein. (A review of Thermoelectric MEMS Devices for Micro-power Generation, Heating and Cooling Applications, Gould and Shammas (2009) Staffordshire University, UK ISBN 978-953-307-027-8, www.intechopen.com; Flexible Micro Thermoelectric Generator based on Electroplated Bi2+xTe3−x, Schwyter, Glatz, Durrer and Hierold, Micro and Nanosystems Dept of Mechanical and Process Engineering, ETH Zurich, Switzerland; Coin-sized coiled-up polymer foil thermoelectric power generator for wearable electronics, Weber, Potje-Kamloth, Detemple, Volklein, and Doll, The 19^{th} European Conference on Solid-State Transducers, http://doi.org/10.1016/j.sna.2006.04.054.)

In addition, it is critical that the state of contraction of the ciliary muscle 52 is continuously monitored in a non-contacting manner. Research results have been encouraging in the field of InfraRed Muscle Contraction Sensor (IR Sensor) technology for sensing the effect of the movement of a muscle on the signal pattern of reflected light.

While EMS, TEG, and IR Sensor technologies exist and have been employed for a variety of applications, they have never been capable of application within the context of presbyopia treatment. Device embodiments disclosed herein provide for the miniaturization and creative packaging solutions that permit the use of EMS, TEG, and IR Sensor technologies at the micro sizes required for the ciliary muscle application, and that enable the use of a new method of stimulating the ciliary muscle 52 to regain vision clarity for the aging eye.

### DESCRIPTION OF EXEMPLARY EMBODIMENTS

### Power Availability in Micro-Size

One embodiment of the disclosed device includes a micro-sized Thermo Electric Generator (“μTEG”) that generates sufficient power for the electrical muscle stimulation (“EMS”) of the ciliary muscle 52, and that may be encapsulated within a contact-lens sized package. The power supply from the μTEG is driven by excess heat produced by the body, whereby heat differences between the eye and the ambient environment may be converted into electricity due in large part to charge carrier diffusion in a conductor. Here, the temperature differential available to drive the μTEG is the difference between the temperature of the eye surface at the cornea (34.5° C. or 307.6K) and the average ambient temperature (22.2° C. or 295.3K), which equals 12.3K. (A Reference for the Human Eye Surface Temperature Measurement, Measurement 2011, Proceedings of the 8^{th} International Conference, Smolenice, Slovakia; Nano-scale Characterization of a Piezoelectric Polymer (PVDF), Sensors, 8, 7359-7368.)

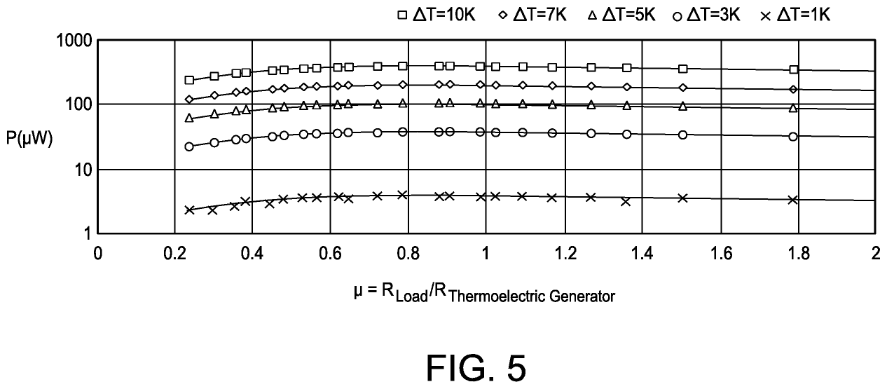

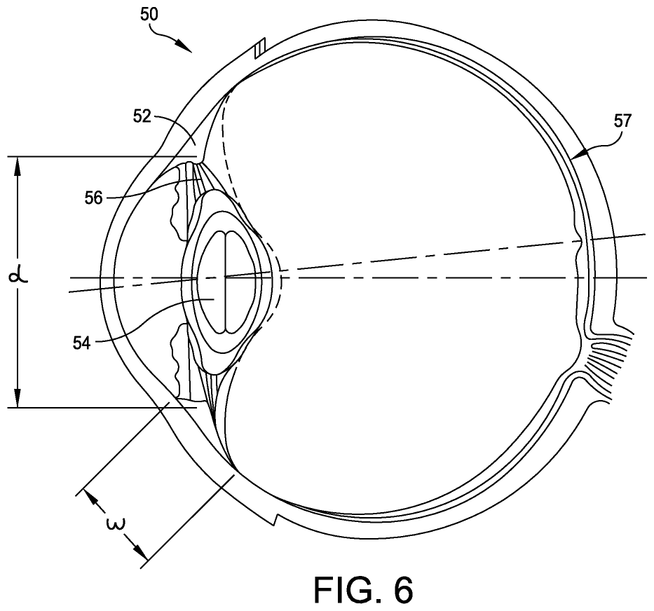

The theoretical power characteristics of an exemplary TEG at varying temperature differentials is published in U.S. Pat. No. 7,629,531 to Starke, summarized in the chart provided in FIG. 5. By comparison, the EMS power requirement for abdominal therapeutic stimulation is well-known in the marketplace. The power requirement for the μTEG disclosed herein is proportional to the ratio of the average surface area of the ciliary muscle 52 divided by the average surface area of the abdominal muscle, multiplied by the known abdominal power requirements. A summary of this calculation is as follows:
The μTEG is driven by the 12.3K temperature differential between the eyeball surface at the cornea, 34.5° C. or 307.6K, and the average ambient temperature, 22° C. or 295.3K. Applying the TEG power characteristics provided in U.S. Pat. No. 7,629,531, and summarized in FIG. 5, for a balanced or matched load in which the resistance of the load equals the resistance of the TEG (μ=1), results in a power generation of 750 μW acting on the ciliary surface area.
As shown in FIG. 6, the average diameter, d, of the ciliary muscle 52 is approximately 12.9 mm. Thus, an average circumference of the ciliary muscle is 40.5 mm, which is multiplied by the average width, w, of the ciliary muscle of 4.5 mm, resulting in an approximate area of the ciliary muscle 52 of 182 mm² or 1.82 cm². Thus, the ciliary power flux available at the μTEG equals the power generation acting on the ciliary surface area, 750 μW, divided by the ciliary surface area, 1.82 cm², or approximately 750 μW/1.82 cm²=412 μW/cm².
The TEG power required for the known EMS for abdominal therapeutic stimulation averages 700 mA at 110 V, or 77 W, applied over the average abdominal area of 620 cm², equaling 0.125 W/cm². This value normalized to the smaller size (1/340) of the ciliary muscle 52 results in a μTEG power requirement of the ciliary muscle 52 of 367 μW/cm².

Thus, the power flux available from the micro-sized TEG at the ciliary muscle 52, or 412 μW/cm², is at least equivalent to the power flux required by the abdominal EMS example normalized to the smaller ciliary muscle 52, or 367 μW/cm² based on a proven therapeutic value.

### Micro-Sized Device Embodiments

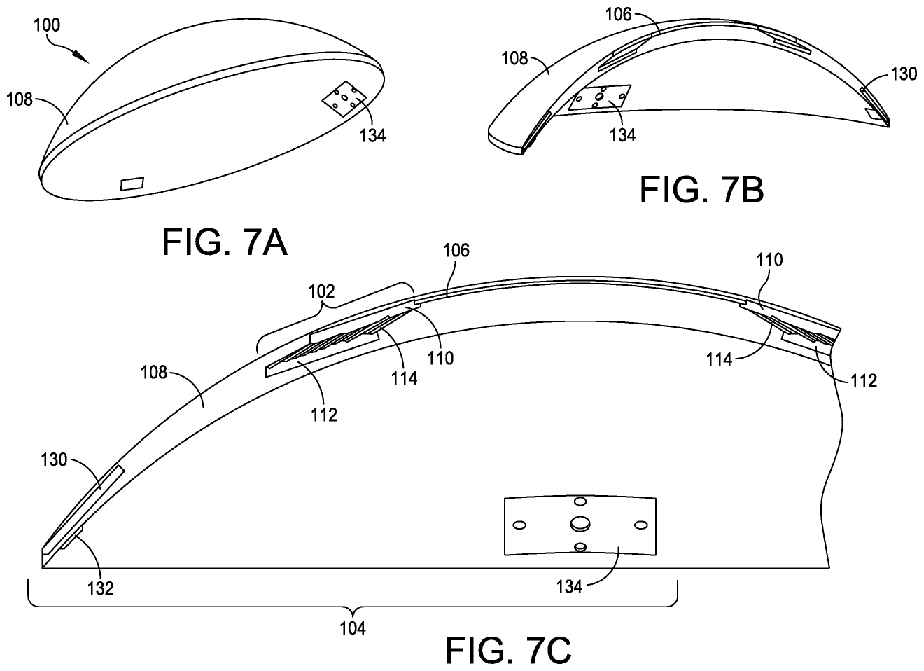

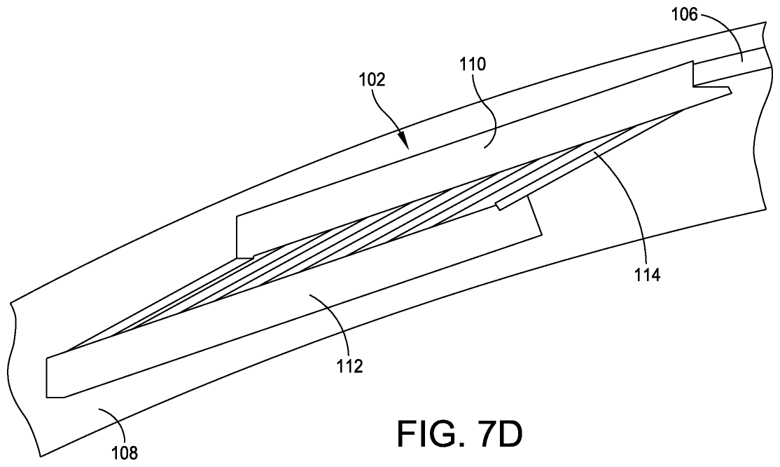

FIGS. 7A-7D illustrate perspective, cross-sectional, partial cross-sectional, and partial views of one embodiment of a contact-lens sized, thermoelectrically-powered device 100 for therapeutic presbyopia vision correction, including a plurality of components and their relationship within a protective enclosure configured to fit flush against the eye, similar to a contact lens. In some embodiments, the thermoelectrically-powered device 100 includes four primary components: a power supply in the form of a micro-sized Thermal Electric Generator (“μTEG”) 102, an electrical disc sub-assembly 104, a dome 106 that optionally secures the shape and position of the μTEG 102, and a silicone hydrogel encapsulate or over-mold 108 that contains or envelops the other components and secures them in relation to one another.

### Power Supply

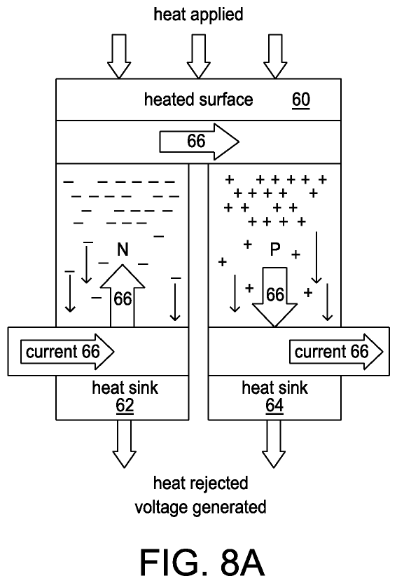

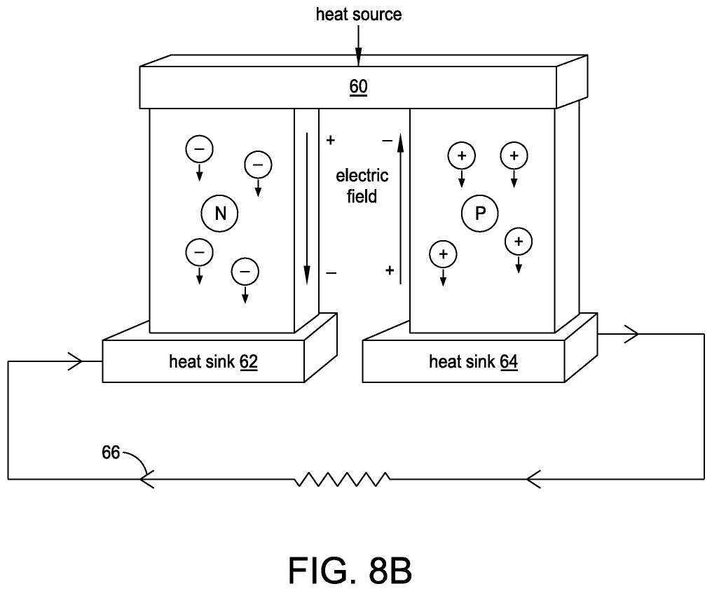

Turning to the individual components of the device 100, and as discussed above, one embodiment of the power supply may take the form of the micro-sized Thermal Electric Generator (μTEG) 102, which comprises a practical application of the Seebeck Effect predicting that positive and negative charged materials that are connected to thermally conductive heat sources, as shown in FIGS. 8A and 8B, will generate electrical current as the heat causes the positive and the negative charge carriers to move from hot to cold.

To demonstrate, in FIGS. 8A-8B, a negatively-doped semiconductor material, N, is in contact with a heated surface 60 at one end and a colder, heat sink 62 at the other end. In parallel, a positively-doped semiconductor, P, is connected to the same hot surface 60 at one end and a second heat sink 64 at the other end. In both the positively- and negatively-doped cases, the charge carriers move from hot to cold in an attempt to establish equilibrium. A resulting current flows from plus to minus in the direction of arrow 66.

The positively- and negatively-doped semiconductors, P and N, are carefully fabricated to have an excess of negatively charged electrons in the N leg of the μTEG and an excess of positively charged “holes” in the P leg of the μTEG. A “hole” is the location of an absent electron in the outer orbit of an atom. Both electrons and holes flow from hot to cold, in both cases moving from an area of high entropy to an area of low entropy, always seeking equilibrium. In addition, the semiconductors, P and N, are made such that although they are highly conductive electrically, they are thermally insulative, which maintains the temperature differential between the heated surface 60 and the heat sinks 62, 64 that drives the whole process.

Returning to FIGS. 7C-7D and in this embodiment, the μTEG 102 may include a cooler top plate 110 and a hotter bottom plate 112, each mechanically adjoined to a stack of semi-flexible, insulative polymer discs 114, upon which the Plus and Minus poles of a thermocouple are printed. In this embodiment, the heat source is the surface temperature of the eyeball, which is in thermal contact with the hotter bottom plate 112. The heat sink is the ambient air at the exposed surface of the eye lens and in thermal contact with the cooler top plate 110. The hotter bottom plate 112 and the cooler top plate 110 may be fabricated from highly thermally conductive materials such as, for example, copper or aluminum. In some embodiments, the top and bottom plates 110, 112 may be parts stamped from thin (0.125 mm) foil. In this embodiment, the insulated, thinner discs (0.050 mm) 114 extend between the hotter bottom plate 112 and the cooler top plate 110. The insulated polymer discs 114 are in thermal contact at each respective end with the hotter bottom plate 112 and the cooler top plate 110. The thinner, insulated polymer discs 114 may be punched from a foil made of an electrically and thermally insulative material. In one embodiment, a polyimide material such as Kapton from DuPont is preferred.

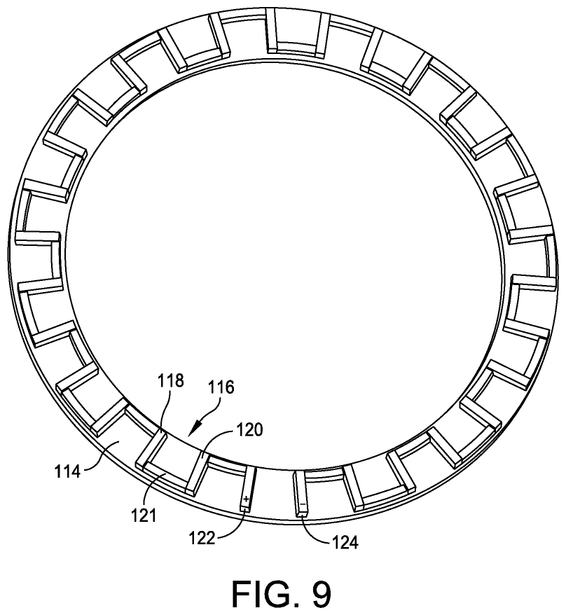

The heat difference between the hot-side bottom plate 112 in contact with the eyeball 50 and the cold-side top plate 110 in contact with the ambient air causes an electric current in a thermocouple, as explained above in reference to FIGS. 8A and 8B. In this embodiment, a series of thermocouples 116 may be printed upon each of the insulated polyamide substrates/foil discs 114 in a pattern shown in FIG. 9. Each of the printed thermocouples 116 may include a negatively-dosed “N” leg 118 and an equivalent but positively-dosed “P” leg 120. The respective negatively- and positively-dosed materials or legs, 118 and 120, may be joined in series by a conductive bridge 121, where the net effect of connecting the series of such thermocouples 116 results in a single “plus” terminal 122 and a single “minus” terminal 124. A sufficient number of foil discs 114, each printed with the thermocouples 116, may be stacked to provide the necessary number of thermocouples 116 to deliver the required current and voltage to the device 100, as discussed above.

In further detail, the N thermoelectric leg 118 and the P thermoelectric leg 120 may be spaced uniformly around the circumference of the insulative disc 114. In this embodiment, the material of each of the N and P semiconductors 118, 120 is based on a compound well-known in the semiconductor industry, bismuth telluride (Bi₂Te₃), taking advantage of the fact that Bismuth is not considered a human carcinogen. The specific compositions of the legs may be altered to enhance the thermoelectric performance, i.e., to “dope” the N-segment 118 with negatively (−) charged electrons and the P-segment 120 with positively (+) charged holes.

In one embodiment, the dimensions of the N and P thermoelectric legs 118, 120 are approximately 0.150 mm×0.150 mm×0.500 mm. Although a number of microfabrication techniques may be utilized in depositing the thermoelectric material onto the insulated substrate disc 114, the method of sputtering (magnetron or plasmatron) is preferable for depositing the thick bismuth-telluride compound on thin substrates such as the substrate disc 114. Similarly, the electrical bridges 121, which may be fabricated from a highly thermally conductive material, such as, for example, gold-plated nickel, may be typically sputtered into position between the thermoelectric legs 118, 120.

In operation, a current flows from each of the P-segments 120 to the N-segments 118 of the thermocouples 116, and when connected in series, as they are shown in FIG. 9, the voltage potential is additive and collected at the plus terminal/electrode 122 and the minus terminal/electrode 124.

Each successively larger foil disc 114 may be constrained in a slight conical form and assembled by thermally connecting each of the discs 114 to the hot and cold plates 112 and 110, respectively, to form a stack of the foil discs 114, each having the respective thermocouples 116 printed thereupon. Thermal adhesive is preferably generously layered onto each of the bottom plate 112 and the top plate 110 and placed in an assembly fixture that accurately positions each disc 114 therebetween. The plus and minus terminals 122, 124 of each of the discs 114 may be connected in series and delivered to a single set of plus and minus terminals as the power outputs of the μTEG assembly 102. In turn, the outputs of the μTEG 102 by be connected with micro diameter wires (not shown) potted onto or embedded into the electric disc sub-assembly 104, as discussed further below.

While the preferred power source is the μTEG 102, discussed above, an alternate embodiment may include a separate component in contact with another part of the user's body to harvest the body heat and produce the power required by the circuitry of the device 100 and transmit that power wirelessly to the electronics on board the over-mold 108. An example might include a headband that harnesses the heat of the forehead or a shirt that harnesses the heat of the torso.

### Electrical Disc Sub-Assembly

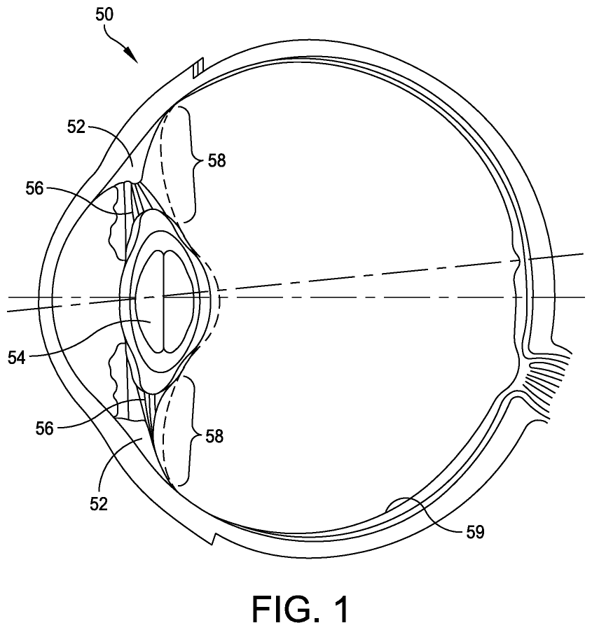

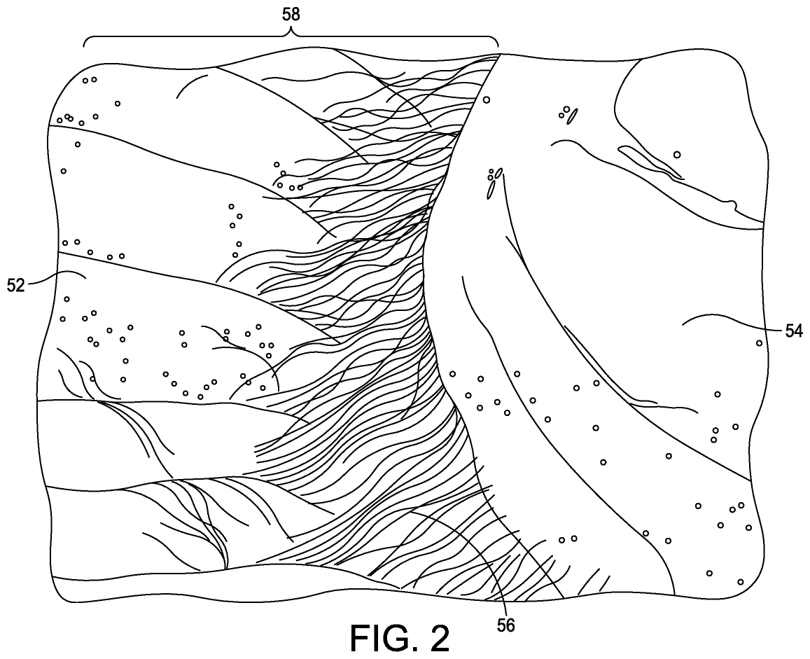

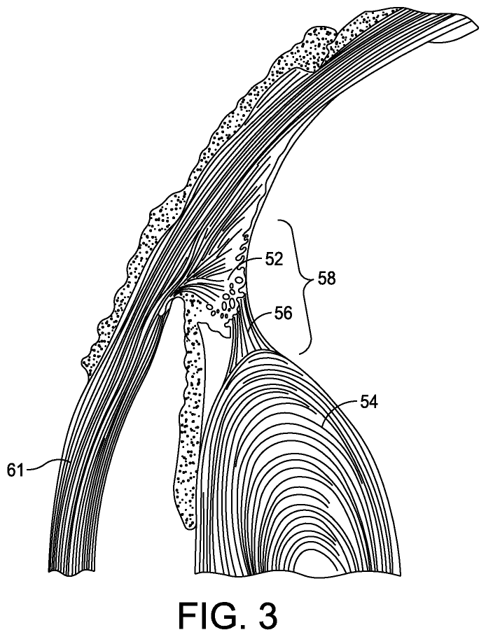

Referring back to FIGS. 1-3 and 4A-4B, embodiments of the therapeutic device for presbyopia vision correction 100 described herein provide a combination of (1) a means to sense the movement or actuation of the sphincter-like ciliary muscle 52; and (2) to electrically stimulate the ring of smooth ciliary muscle fibers responsible for changing the shape of the lens 54 of the eye 50 to achieve near- and far-vision accommodation.

As shown in FIGS. 1-3 and 4A-4B, the suspensory ligaments or zonules 56 connect the ciliary muscle 52 to the periphery of the lens 54. When the ciliary muscle 52 is relaxed, the ligaments 56 become tauter and the lens 54 is stretched thin or flat, enabling the lens 54 to focus on distant objects. Conversely, when the ciliary muscle 52 is contracted, the suspensory ligaments 56 become less taut, and the lens 54 becomes rounder so that it can focus on objects that are nearby.

Embodiments of the therapeutic device 100 described herein sense the motion or actuation of the ciliary muscle 52 during the contraction step and further stimulate the ciliary muscle 52 with minute EMS current/voltage to further contract and strengthen the muscle 52, which, in turn, further relaxes the zonules 56 to improve nearby vision. The same sensor senses the relaxation of the ciliary muscle 52 and shuts down the current/voltage supply, returning the lens 54 to its default position of distance vision. In combination, this exercise improves the strength of the ciliary muscle 52 and the elasticity of the lens 54, such that the natural accommodation of the eye's near and far focus is improved and, therefore, the need for the device 100 diminishes over time.

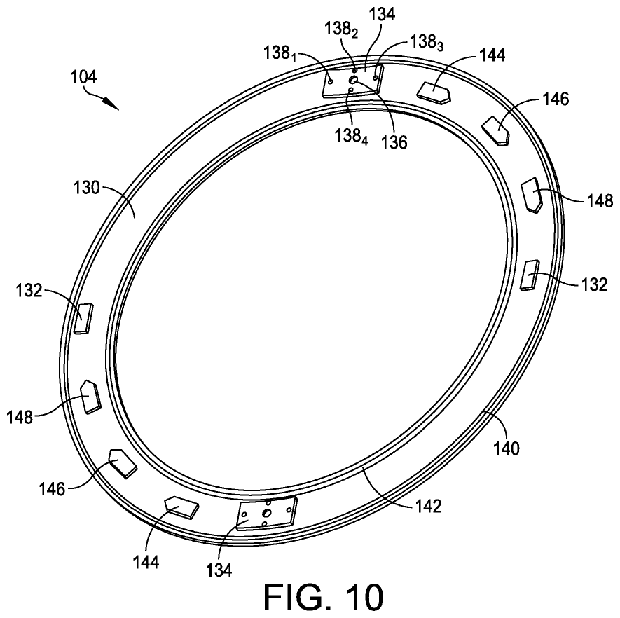

Returning to FIG. 7A-7C, embodiments of the therapeutic device 100 may include an electrical disc sub-assembly 104 responsible for the sensing and motion control/stimulation operations with respect to the ciliary muscle 52, described above. In one embodiment detailed in FIG. 10, the electrical disc sub-assembly 104 may include a flexible polyimide disc or circuit board 130, an electrode 132, and an IR sensor 134. In this embodiment, the non-conductive, flexible polyimide disc or circuit board 130 may support a number of electrodes 130, sensors 134, and a plurality of power/logic circuits of the sub-assembly 104 in a manner similar to that shown in FIG. 10.

The flexible polyimide circuit board 130 may be formed from an electrically and thermally insulative material such as, for example, a 100-micron thick polyimide film such as Kapton from DuPont. Kapton uniquely can be punched as a flat disc, metallized with electrically conductive traces, adhesive coated to secure components, and thermo-formed into a truncated cone shape as shown in the cross-sectional view of FIG. 7B.

Embodiments of the electrical disc sub-assembly 104 may include a plus (+) bus 140 and a minus (−) bus 142, each powered by the output terminals of the μTEG 102, i.e., the plus (+) terminal 122 and the minus (−) terminal 124, through the micro diameter wires (not shown) discussed above. The circumferential buses 140, 142 may power all of the electronics of the electrical disc sub-assembly 104 on the circuit board 130, detailed below.

In this embodiment, the electrode 132 may conduct the voltage and the current from the μTEG power supply 102, discussed above, to the ciliary muscle 52. The electrode 132 may take the form of a polished aluminum or stainless-steel pad interconnected to the outputs of the μTEG 102 by the micro diameter wires (not shown) potted onto or embedded into the electrical disc/busses 140, 142.

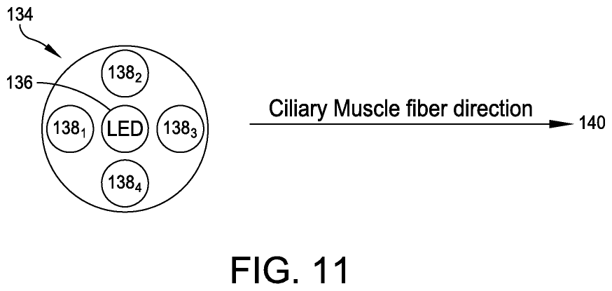

The IR sensor 134 may deliver an on/off logic signal to the electrode 132 and include a sensing mechanism for detecting the change of state of the ciliary muscle 52 between the relaxed state 64, shown in FIG. 4A, and the contracted state 66, shown in FIG. 4B. In one embodiment, the detection or sensing means may comprise one or more non-contacting InfraRed Muscle Contraction sensors, or IR sensors. (An IR Muscle Contraction Sensor, Raghav Subramaniam, Thesis, Cornell University; An electro-optical muscle contraction sensor, Alsessio Chianura and Mario Giardini, Med Biol Comput 48: 731-734.) As schematically detailed in FIG. 11 and in one embodiment, the IR sensor 134 may consist of an IR LED emitter 136 surrounded by a phototransistor array including four equally spaced IR phototransistors 138₁₋₄, two arranged parallel and two arranged perpendicular to the direction of the fibers of the ciliary muscle 52, depicted by arrow 140.

In this embodiment, the light emitted from the IR LED emitter 136 reflects off the target ciliary muscle 52 despite the muscle 52 being positioned under a sclera layer 57 of the eyeball 50 (FIG. 1), the thickness of which is approximately 550 microns, and the reflected light is sensed by the phototransistor array 138₁₋₄. Muscle contraction is detected by measuring a change that occurs between light that is scattered in the directions that are parallel to and perpendicular to the muscle fibers. Because light scattering is dominated by the presence of blood in the ciliary muscle 52 and because, during contraction, the muscle 52 undergoes blood depletion, the contraction of the ciliary muscle 52 can be detected as a decrease in the optical absorption of the muscle.

Returning to FIG. 10 and in this embodiment, the electrical disc sub-assembly 104 may include two IR sensors 134, each positioned on the periphery of the flexible disc/circuit board 130, with the pattern of the parallel phototransistors 138₁₋₄ surrounding the IR LED emitter 136. The parallel phototransistors 138_{1, 3} are configured to collect the light scattered parallel to the fibers of the ciliary muscle 52, and the perpendicular phototransistors 138_{2,4} are configured to collect the light scattered perpendicular to the fibers of the ciliary muscle 52, as detailed in FIG. 11.

Additional control and logic components may be required by the IR sensors 134 and the electrodes 132 of the electrical disc sub-assembly 104. In further detail, one embodiment of the sub-assembly 104 may require at least three control devices comprising interconnecting integrated circuit (“IC”) or chip components for amplification of the output of the IR sensors 134 and for the switching of logic states. While these components are shown schematically in FIG. 10, they may be printed upon the circuit board/flexible electronic disc 130 directly or implemented in any appropriate manner.

In one embodiment, one or more state detection logic controllers 144 may be configured to respond to the output of the IR sensors 134. In this embodiment, the logic controller 144 may be a switching circuit that compares the output electronic signal wave shape from each of the four phototransistors 138₁₋₄ of each of the IR sensors 134 with one or more known/anticipated wave formats associated with the optical absorption of the ciliary muscle 52 in either the relaxed state 64 or the contracted state 66, as appropriate. Using this comparison, the state detection logic controller 144 may derive and deduce whether or not the ciliary muscle 52 has been activated for contraction by parasympathetic nerve signals from the brain, and track the change of state of the ciliary muscle 52 between the relaxed state 64 and the contracted state 66 (FIGS. 4A-4B).

The change of state of the ciliary muscle 52 detected by the state detection logic controller 144 may provide an output to a switching and amplification circuit 146, thereby opening the circuit 146 to the plus (+) and minus (−) buses 140, 142 for muscle stimulation to induce further contraction of the ciliary muscle 52 or closing the circuit 146 to allow relaxation of the ciliary muscle 52. That is, when the switching and amplification circuit 146 opens, the current from the plus (+) bus 140 to the minus (−) bus 142 is amplified and conducted directly to the electrodes 132, which excite the ciliary muscle 52 to contract further than it naturally could or would without stimulation. When the switching and amplification circuit 146 closes, the current from the plus (+) bus 140 to the minus (−) bus 142 is restricted/terminated, terminating the muscle stimulation and allowing the ciliary muscle 52 to return to the relaxed state 64. The switching and amplification circuit 146 may also be programmed to deliver a cyclical wave shape of current to the electrodes 132 and, in turn, to the ciliary muscle 52 for the purpose of exercising (e.g., cyclically contracting and relaxing) the special ciliary muscle 52 without inducing fatigue. A signal conditioning circuit 148 may be configured to deliver maximum power to the electrodes 132, as necessary.

While the preferred means to detect movement of the smooth fibers of the ciliary muscle 52 is via the non-contacting IR sensors 134 that provide an output to the state detection logic controller 144, discussed above, an alternative embodiment may employ a Piezo-Polymer film layer, or a PVDF film layer, that specifically bends in response to ciliary muscle contraction and emits a microampere signal to an on/off logic switching circuit such as the state detection logic controller 144. (Wearable Systems based on PVDF Sensors in Physiological Signals Monitoring, FerroElectrics, Vol 500, 2016—Issue 1 Yi Xi, et. Al. 14 Oct. 2016.) In this embodiment, the space occupied by the circuit board 130 may be reallocated to allow for the PVDF film layer. A continuous circumferential band or film of PVDF would be in the best position to measure movement of the contracting ciliary muscle 52.

### Dome

In relation to FIGS. 7B-7C and in one embodiment, a redundant plastic dome 106 may secure the shape and position of the μTEG assembly 102. In this embodiment, the dome 106 may be an optically clear, gas-permeable, plano lens that is generally placed such that the insulated polymer rings/discs 114 of the μTEG 102 are positioned within a boundary defined by the hydrogel envelope or encapsulate 108. The dome 106 provides optional shaping and positioning, but is not necessary for functionality of the μTEG 102.

### Encapsulate

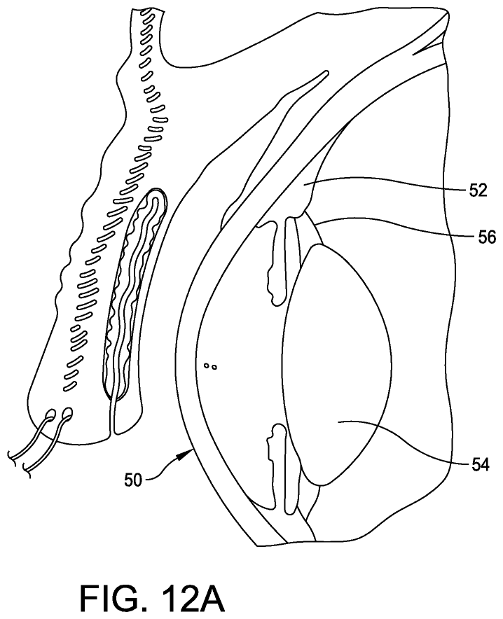

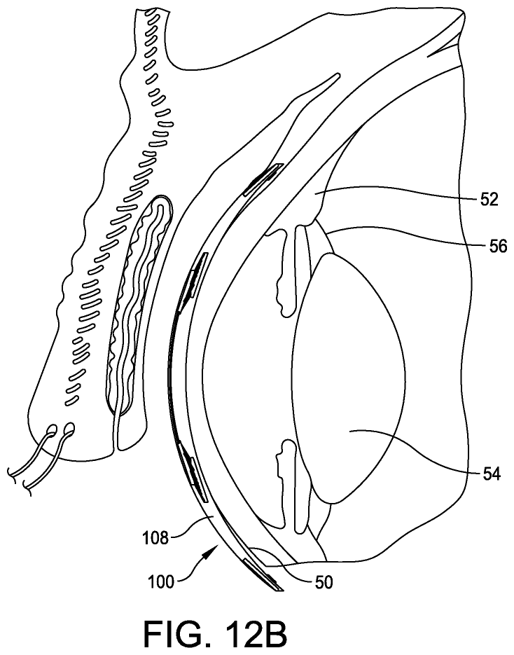

In one embodiment shown in FIGS. 7A-7D, the μTEG 102 and the electrical disc sub-assembly 104 are placed into a contact-lens-like package such as the encapsulate/over-mold 108, which is worn against or flush with the eyeball 50 in a manner similar to a contact lens, as shown to scale in FIGS. 12A-12B. In this embodiment, the encapsulate 108 may be a hydrogel over-mold surrounding the components of the therapeutic device, including the components of the μTEG 102, the dome 106, and the electrical disc sub-assembly 104, and securing them in an appropriate relationship to one another.

### Method

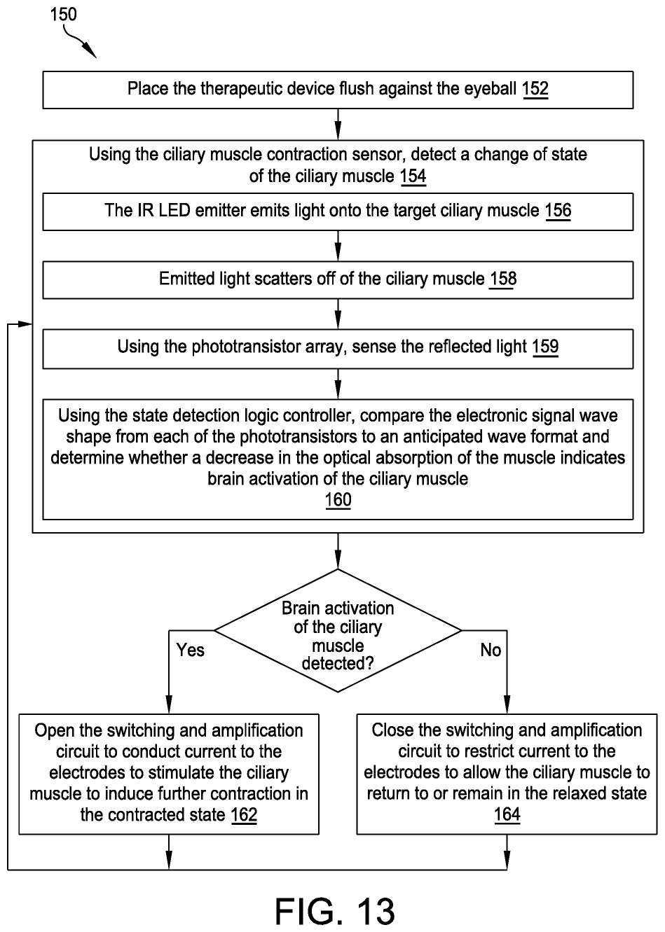

FIG. 13 provides a flowchart depicting an exemplary method (150) of using an embodiment of the device 100 to therapeutically stimulate the ciliary muscle 52 for presbyopia vision correction. In one embodiment, the method (150) initiates with placing the therapeutic device 100 against the eyeball 50 similar to the placement of a contact lens (152), as shown in FIGS. 12A-12B. Once positioned, the ciliary muscle contraction sensor, or the IR sensor 134, as well as the state detection logic controller 144 may be employed to detect a change of state of the ciliary muscle 52 (154). This state detection (154) may involve operating the IR LED emitter 136 of the IR sensor 134 to emit light onto the target ciliary muscle (156). The emitted light that reflects or scatters off of the ciliary muscle 52 (158) is then sensed by the phototransistor array 138₁₋₄ (159) and output signal waves for each of the phototransistors 138₁₋₄ are provided to the state detection logic controller 144. The logic controller 144 may compare an electronic signal wave shape from each of the phototransistors 138₁₋₄ to an anticipated wave format, as discussed above, to determine whether a decrease in the optical absorption of the ciliary muscle indicates brain activation of the ciliary muscle 52 (160).

If brain activation of the ciliary muscle 52 is detected, the state detection logic controller 144 may provide an open input to the switching and amplification circuit 146, causing the circuit 146 to open to the plus (+) and minus (−) busses 140, 142 such that the current from the plus (+) bus 140 to the minus (−) bus 142 is amplified and conducted to the electrodes 132, thereby stimulating or exciting the ciliary muscle 52 and inducing further contraction of the ciliary muscle 52 in the contracted state 66 for nearby focus (162). If brain activation of the ciliary muscle 52 is absent, the logic controller 144 may provide a close input to the switching and amplification circuit 142, causing the switching and amplification circuit 146 to close or remain closed, such that the current from the plus (+) bus 140 to the minus (−) bus 142 is restricted and the ciliary muscle does not receive stimulation from the electrodes 132, allowing the ciliary muscle 52 to return to or remain in the relaxed state 64 for far focus (164). Detecting brain activation of the ciliary muscle 52 may be continuous, thereby allowing the device 100 to exercise the eye in accordance with the eye's natural progression into and out of near and far vision.

In another embodiment, as discussed above, the switching and amplification circuit 146 may also be programmed as appropriate and/or desired to deliver a cyclical wave shape of current to the electrodes 132 in order to purposefully exercise (e.g., cyclically contracting and relaxing) the ciliary muscle 52 without inducing fatigue. In other embodiments, the switching and amplification circuit 146 may be programmed as appropriate to achieve any desired therapeutic benefits.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. A therapeutic device for presbyopia vision correction of a human eyeball having a ciliary muscle, a lens, and a plurality of suspensory ligaments connecting the ciliary muscle to a periphery of the lens, the therapeutic device comprising:
an over-mold configured to fit flush against the eyeball;
a micro power supply encapsulated by and secured within the over-mold; and
an electrical disc sub-assembly powered by the micro power supply and encapsulated by and secured within the over-mold, the electrical disc sub-assembly configured for:
detecting an activation of the ciliary muscle from a relaxed state in which the plurality of the suspensory ligaments are pulled taut and the lens is stretched into a flat shape for distant focus toward a contracted state in which the plurality of the suspensory ligaments relax and the lens moves into a rounded shape for nearby focus;
when the activation of the ciliary muscle is detected, providing an electrical muscle stimulation (EMS) to the ciliary muscle to cause the ciliary muscle to contract further, thereby further relaxing the plurality of the suspensory ligaments to improve the nearby focus; and
when the activation of the ciliary muscle is not detected, terminating the EMS to allow the ciliary muscle to return to the relaxed state, thereby pulling the plurality of the suspensory ligaments taut and returning the lens to the flat shape for the distant focus.

2. The therapeutic device of claim 1, wherein the over-mold has a size and a shape of a contact lens.

3. The therapeutic device of claim 2, wherein the over-mold is formed from a hydrogel.

4. The therapeutic device of claim 1, wherein the micro power supply comprises a micro thermoelectric generator (μTEG).

5. The therapeutic device of claim 4, wherein the μTEG comprises:
a cool top plate in thermal contact with an ambient environment;
a hot bottom plate in thermal contact with a surface of the eyeball; and
a stack of insulative polymer discs, each of the insulative polymer discs having a top end in thermal contact with the hot top plate and a bottom end in thermal contact with the cool bottom plate, wherein:
a thermocouple is printed upon each of the insulative polymer discs, the thermocouple having a positive and a negative terminal; and
a heat differential between the cool top plate and the hot bottom plate generates an electric current that flows between the positive and the negative terminals of the thermocouple.

6. The therapeutic device of claim 5, wherein each of the insulative polymer discs is formed of a polyimide foil.

7. The therapeutic device of claim 5, further comprising a plastic dome configured to secure a shape and a position of the μTEG within the over-mold.

8. The therapeutic device of claim 1, wherein the electrical disc sub-assembly comprises:
an electrode;
an infrared (IR) sensor configured to sense a scattered light reflected off of the ciliary muscle;
a state detection logic controller configured to receive an electronic signal wave from the IR sensor that is representative of the scattered light reflected off of the ciliary muscle, compare the electronic signal wave from the IR sensor to an anticipated signal wave format, and determine whether a decrease in an optical absorption of the ciliary muscle indicates the activation of the ciliary muscle; and
a switching and amplification circuit configured to receive an input from the state detection logic controller and, when the activation of the ciliary muscle is detected, to conduct a current supplied by the micro power supply to the electrode such that the electrode provides the EMS to the ciliary muscle.

9. The therapeutic device of claim 8, wherein the IR sensor comprises:
an IR LED emitter for emitting a light onto the ciliary muscle; and
a plurality of phototransistors for sensing the scattered light reflected off of the ciliary muscle, wherein one or more of the phototransistors are positioned parallel to a plurality of fibers of the ciliary muscle and one or more of the phototransistors are positioned perpendicular to the plurality of the fibers of the ciliary muscle.

10. A therapeutic device for exercising a ciliary muscle of a human eyeball, comprising:
an encapsulate configured to fit flush against the eyeball, the encapsulate enveloping:
a micro thermoelectric generator (μTEG) configured to generate power from a temperature differential between a surface temperature of the eyeball and an ambient temperature of an ambient atmosphere; and
an electrical disc sub-assembly coupled with and powered by the μTEG, the electrical disc sub-assembly configured to provide an electrical muscle stimulation (EMS) to the ciliary muscle.

11. The therapeutic device of claim 10, wherein the μTEG comprises:
a cool thermally conductive plate in thermal contact with the ambient atmosphere;
a hot thermally conductive plate in thermal contact with the eyeball; and
a stack of thermally insulated discs, each of the thermally insulated discs in thermal contact with the cool thermally conductive plate and the hot thermally conductive plate, wherein:
each of the thermally insulated discs supports a series of thermocouples, each including a negatively dosed leg having a plurality of negative charge carriers and a positively dosed leg having a plurality of positive charge carriers, the negatively dosed and the positively dosed legs connected by an electrical bridge; and
the temperature differential between the hot thermally conductive plate and the cool thermally conductive plate causes the plurality of the negative and the positive charge carriers to generate an electrical current through the series of the thermocouples of each of the thermally insulated discs to a power output as the pluralities of the negative and the positive charge carriers move away from the hot thermally conductive plate toward the cool thermally conductive plate.

12. The therapeutic device of claim 10, wherein the electrical disc sub-assembly comprises:
a disc circuit board supporting a positive bus and a negative bus, each electrically coupled with the power output of the μTEG;
an electrode electrically coupled across the positive and the negative busses;
a switching and amplification circuit configured to conduct a cyclical current flow from the power output of the μTEG to the positive and the negative busses across the electrode, thereby causing the electrode to provide the EMS to the ciliary muscle in a cyclical manner that moves the ciliary muscle cyclically between a relaxed state for distant focus and a contracted state for nearby focus.

13. The therapeutic device of claim 10, the electrical disc sub-assembly further configured to detect a brain activation of the ciliary muscle and provide the EMS to the ciliary muscle when the brain activation is detected.

14. The therapeutic device of claim 13, wherein the electrical disc sub-assembly comprises:
a disc circuit board supporting a positive bus and a negative bus, each electrically coupled with a power output of the μTEG;
an electrode electrically coupled across the positive and the negative busses;
a switching and amplification circuit for controlling a current flow from the positive bus to the negative bus across the electrode;
an infrared (IR) sensor configured to emit a light onto the ciliary muscle and measure the light that is scattered by ciliary muscle; and
a state detection logic controller in communication with the IR sensor and the switching and amplification circuit, the state detection logic controller configured to:
based upon the light that is scattered by the ciliary muscle, detect the brain activation of the ciliary muscle; and
when the brain activation of the ciliary muscle is detected, open the switching and amplification circuit to conduct the current flow across the electrode and provide the EMS to the ciliary muscle.

15. The therapeutic device of claim 14, the IR sensor comprising:
an IR emitter configured to emit the light onto a plurality of fibers of the ciliary muscle; and
a phototransistor array positioned about a periphery of the IR emitter, the phototransistor array configured to measure one or more electronic signal wave shapes representing the light that is scattered by the ciliary muscle in a parallel direction and in a perpendicular direction to the plurality of the fibers of the ciliary muscle.

16. The therapeutic device of claim 15, the phototransistor array comprising:
at least one phototransistor positioned parallel to a direction of the plurality of the fibers of the ciliary muscle and configured to collect the light that is scattered in the parallel direction; and
at least one phototransistor positioned perpendicular to the direction of the plurality of the fibers of the ciliary muscle and configured to collect the light that is scattered in the perpendicular direction.

17. A method of correcting presbyopia in a human eyeball having a ciliary muscle, a lens, and a plurality of zonules connecting the ciliary muscle to a periphery of the lens, the method comprising:
positioning a therapeutic device for presbyopia vision correction flush against the eyeball, the therapeutic device having an encapsulate encasing a micro thermoelectric generator (μTEG) and an electrical disc sub-assembly electrically coupled with the μTEG, the electrical disc sub-assembly having a switching and amplification circuit communicatively coupled with at least one electrode;
operating the μTEG to generate power from a temperature differential between a surface temperature of the eyeball and an ambient temperature of an ambient atmosphere; and
conducting, via the switching and amplification circuit, a current flow from a power output of the μTEG to the at least one electrode to provide an electrical muscle stimulation (EMS) to the ciliary muscle.

18. The method of claim 17, wherein the current flow comprises a cyclical current flow causing the ciliary muscle to cyclically move between a relaxed state in which the plurality of the zonules are pulled taut and the lens is stretched into a flat position for distant focus and a contracted state in which the plurality of the zonules relax and the lens moves into a rounded position for nearby focus.

19. The method of claim 17, further comprising:
prior to the conducting the current flow, detecting, via a ciliary muscle contraction sensor in communication with a state detection logic controller of the electrical disc sub-assembly, a brain activation of the ciliary muscle from a relaxed state in which the plurality of the zonules are pulled taut and the lens is stretched into a flat position for distant focus toward a contracted state in which the plurality of the zonules relax and the lens moves into a rounded position for nearby focus.

20. The method of claim 19, wherein the detecting the brain activation of the ciliary muscle comprises:
emitting, via an infrared (IR) LED emitter of the ciliary muscle contraction sensor, a light onto the ciliary muscle;
measuring, via a phototransistor array of the ciliary muscle contraction sensor, the light scattered by the ciliary muscle;
receiving, at the state detection logic controller from the phototransistor array, one or more electronic signal wave shapes representing the light scattered by the ciliary muscle;
comparing, via the state detection logic controller, the one or more of the electronic signal wave shapes against one or more anticipated wave formats associated with an optical absorption of the ciliary muscle; and
determining, via the state detection logic controller and based upon the comparing the one or more of the electronic signal wave shapes against the one or more of the anticipated wave formats, that a change in the optical absorption of the ciliary muscle indicates the brain activation of the ciliary muscle.
